(19) United States
(12) Patent Application Publication
(10) Pub. No.: US 2006/0193908 A1
(43) Pub. Date: Aug. 31, 2006

(54) Extended release formulations of poorly soluble antibiotics

(76) Inventors: Beth A. Burnside, Bethesda, MD (US); Colin Rowlings, Potomac, IN (US); Sandra E. Wassink, Frederick, MD (US); Donald J. Treacy JR., Woodbine, MD (US); John J. Koleng, Austin, TX (US)

Correspondence Address: INNOVAR, LLC, P O BOX 250647, PLANO, TX, 75025, US

(21) Appl. No.: 11/267,943

(22) Filed: Nov. 4, 2005

Related U.S. Application Data
(60) Provisional application No. 60/626,277, filed on Nov. 9, 2004.

(51) Int. Cl. A61K 31/7048; A61K 9/20

(52) U.S. Cl. 424/464; 514/29

(57) ABSTRACT

An extended release pharmaceutical compressed composition and dosage form comprising poorly water soluble macrolide antibiotic, surfactant and non-lipophilic, non-polymeric excipient is disclosed. The composition releases the macrolide antibiotic over an extended period of time, generally at least over 12 hours, even in the absence of a release rate-retarding polymer, release rate-retarding coating or release rate-retarding lipophilic excipient. The composition is suitable for once daily or twice daily oral administration for the treatment of many different types of bacterial infections. One embodiment of the compressed composition includes a drug-containing granular composition and a binding composition, wherein the two are mixed together and then compressed into a tablet or pill. The surfactant is in admixture with or coated onto the macrolide antibiotic, and it can be included in the granular composition and/or the binding composition. The non-polymeric, non-lipophilic excipient is included in the granular composition and/or the binding composition.

### CROSS-REFERENCE TO EARLIER FILED APPLICATION

The present application is a continuation-in-part of and claims the priority of U.S. Provisional Application for Patent No. 60/626,277 filed Nov. 9, 2004, the entire disclosure of which is hereby incorporated by reference

### FIELD OF THE INVENTION

The invention relates to a pharmaceutical composition that provides an extended release of antibiotic drug. The invention also relates to methods of making such pharmaceutical compositions, and methods of treating a bacterial infection in a host with such pharmaceutical compositions.

### BACKGROUND OF THE INVENTION

Azithromycin, clarithromycin, dirithromycin, erythromycin, troleandomycin, tylosin, and other macrolide antibiotics are used in treating bacterial infections.

The United States Pharmacopoeia/National Formulary (hereinafter “the USP 27/NF 22”) lists clarithromycin as practically insoluble in water.

Clarithromycin is indicated for the treatment of bacterial infections such as pharyngitis, tonsillitis, acute maxillary sinusitis, acute exacerbation of chronic bronchitis, pneumonia, uncomplicated skin and skin structure infections, and disseminated mycobacterial infections.

Macrolide antibiotics are typically administered orally by way of immediate release dosage forms or solutions. Immediate release dosage forms comprise a range of different commonly used excipients that usually do not cause an extended release of the drug. Instead, they are adapted to disintegrate or dissolve rapidly after oral administration. In other words, a dosage form including such excipients or mixtures thereof will generally provide a rapid release rather than an extended release of drug.

Macrolide antibiotics are also available in extended release solid oral dosage forms. However, those dosage forms include excipients adapted to prolong the release of drug from the dosage form. Matrix dosage forms are designed to dissolve or to disintegrate over an extended period of time. Pulsatile dosage forms are designed to provide sequential periodic bolus releases of drug over an extended period of time. Osmotic pumps and other coated dosage forms are designed to provide a substantially continuous release of drug over an extended period of time through a preformed passageway in a wall surrounding a drug-containing core, wherein the wall retains its physical integrity during drug release.

For the most part, uncoated compressed extended release dosage forms are also matrix dosage forms containing one or more release rate-retarding polymers or slowly disintegrating lipophilic excipients such as waxes.

Extended (controlled, sustained, prolonged or slow) release formulations of erythromycin, clarithromycin, and other macrolide antibiotics are well known and are disclosed in many patent publications. A wide range of different materials is suitable for use in such formulations. The formulations vary widely in their composition, requirements, and performance. The known extended release formulations require a release rate-retarding polymer, a lipophilic excipient, or a combination of other features in order to effect the extended release.

Clarithromycin is commercially available under the trademark BIAXIN® XL in extended release form, a 500 mg oral tablet. The product provides extended release of clarithromycin by using a release rate-retarding polymer. The polymer is required in order to achieve an extended release of the drug.

Other extended release formulations for poorly soluble antibiotics contain waxes, fats, and/or fatty acids. These formulations require a slow disintegrating lipophilic excipient. In some such formulations wherein the excipient applied is molten wax the formulation methodologies can be hazardous and can result in a dosage form that contains a limited amount of active ingredient.

Similarly, extended release formulations containing a release rate-retarding polymer have many disadvantages. Natural polymers exhibit substantial variation from lot to lot that can effect their physicochemical properties and influence their function in the extended release formulation. Each rate-retarding polymer will exhibit variations in hydration rate and drug diffusion depending upon process used to prepare the extended release formulation. The functionality of the rate-retarding polymer is influenced by common variables such as the particle size distribution of the material, the moisture content of the polymer, and the process and means of production of the material. Rate-retarding polymers are also known to exhibit a time dependent aging phenomenon that impacts the performance of the formulation. A need remains for extended release formulations that do not rely upon release rate-retarding polymer(s) or release rate-retarding lipophilic excipients.

### SUMMARY OF THE INVENTION

Applicants have found that the combination of a macrolide antibiotic, a surfactant, and a non-lipophilic, non-polymer excipient(s) can be used to control erosion of a compressed solid dosage form, i.e., erosion of the surface of the compressed dosage form. Applicants have further found that this controlled erosion is markedly improved when the formulation includes both a non-lipophilic, non-polymer excipient, e.g. a monosaccharide and/or a disaccharide, and a high HLB surfactant. This combination of ingredients modifies the physical aspects of the compressed dosage form thereby allowing for extended release of the macrolide antibiotic (such as clarithromycin or erythromycin) by retarding its release. Owing to their amphiphilic nature, the high HLB surfactants used in the present invention serve the dual functions of promoting the wettability of the compressed dosage form surface in contact with bodily fluids within the G.I. tract, and simultaneously retarding the release of the macrolide from the compressed dosage form.

The improved controlled erosion and extended release of the instant invention are counterintuitive in view of the polymer-dependent Biaxin XL product and formulations using hydrophobic materials to achieve extended release. Moreover, surfactants are generally known in the art to be instantaneous or rapid release ingredients, and high HLB surfactants typically tend to increase the dissolution rate of water-insoluble excipients and materials. Thus, the artisan would not have expected that the use of a high HLB surfactant in conjunction with non-lipophilic, non-polymer excipients would result in a controlled erosion and the concomitant extended release of the active ingredient.

The present invention includes a compressed solid oral dosage form that provides an extended release of macrolide antibiotic. The dosage form comprises a therapeutically effective amount of macrolide antibiotic, a surfactant, a non-lipophilic non-polymeric excipient and optionally one or more pharmaceutically acceptable excipients. The antibiotic is released over an extended period of time even in the absence of a release rate-retarding polymer or lipophilic release rate-retarding excipient. The dosage form erodes and/or dissolves slowly over an extended period of time thereby releasing the antibiotic over an extended period of time.

One aspect of the invention provides a compressed extended release solid oral pharmaceutical composition comprising:

a macrolide antibiotic;

one or more surfactants;

one or more non-lipophilic, non-polymeric excipients; and

optionally one or more pharmaceutically acceptable excipients, wherein the surfactant and the non-lipophilic, non-polymeric excipient cooperate to provide an extended release of macrolide antibiotic when the composition is exposed to an aqueous environment of use.

Specific embodiments of the invention include those wherein: 1) the composition comprises a granular solid; 2) the surfactant is selected from the group consisting of sodium lauryl sulfate, Aerosil OT, cholic acid, and salts of cholic acids and the like or a combination thereof; 3) the macrolide antibiotic is, azithromycin, clarithromycin, dirithromycin, erythromycin, oleandomycin, roxithromycin, troleandomycin, or a derivative thereof 4) the surfactant is present in an amount of up to about 10% wt. of the composition; 5) the non-lipophilic, non-polymeric excipient is present in an amount of about 20-60% wt. of the composition; 6) the macrolide antibiotic is present in an amount of about 30-80% wt. of the composition; 7) the composition is substantially free of a release rate-retarding polymer; 8) the composition is substantially free of a release rate-retarding lipophilic excipient; 9) the non-lipophilic non-polymeric excipient is selected from the group consisting of lactose, maltose, dextrose, sucrose, fructose, non-polymeric carbohydrate, alpha-hydroxy acid, polyols (such as mannitol, sorbitol, xylitol, and the like), dicalcium phosphate, calcium sulfate, or a combination thereof, with lactose and the non-lipohilic, non-polymeric surfactant sodium lauryl sulfate being most preferred; 10) the surfactant has an HLB (hydrophilic—lipophilic balance) number of at least 10; 11) the surfactant is non-polymeric; 12) the non-lipophilic, non-polymeric excipient is a monosaccharide or disaccharide; 13) the composition is substantially free of pharmaceutical polymers, waxes, fats, fatty acids, and/or fatty alcohols; 14) the composition provides a release of clarithromycin substantially similar to that provided by the composition disclosed in U.S. Pat. No. 6,010,718; 15) the composition is substantially free of a release rate-retarding polymer; 16) the compressed composition is made by mixing together and then compressing a granular composition and a binding composition; 17) the compressed composition further comprises a glidant present in an amount of about 0.01-5% by weight of the composition; 18) the compressed composition further comprises a lubricant present in an amount of about 0.01-5% by weight of the composition; 19) the composition further comprises a wetting agent present in an amount of about 0.1-10% by weight of the composition; and/or 20) the compressed composition has a hardness of about 5-40 Kp.

Another aspect of the invention provides a method of treating a bacterial infection in a subject, the method comprising the step of orally administering to the subject one or more unit doses of an extended release dosage form or composition as described herein whereby the macrolide antibiotic is delivered to the subject over an extended period of time.

Specific embodiments of the invention include those wherein: 1) the one or more unit doses are administered on a once or twice daily basis; 2) the subject is administered one or more dosage forms per day for a period of 2-14 days; 3) the subject is administered about 100-4000 mg of the macrolide antibiotic daily; 4) a unit dose comprises one or two dosage forms; 5) the subject is administered one to two unit doses per day for a period of at least 2 days; and/or 6) the subject is administered 100 mg to 4 g of macrolide antibiotic per day.

Other features, advantages, and embodiments of the invention will become apparent to those skilled in the art by the following description, and accompanying examples.

### BRIEF DESCRIPTION OF THE FIGURES

FIGS. 1-13 depict dissolution profiles for the exemplary dosage forms of Examples 1-13 as compared to a reference sample.

### DETAILED DESCRIPTION OF THE INVENTION

The United States Pharmacopoeia/National Formulary (hereinafter “the USP 27/NF 22”) defines the relative solubilities of water soluble compounds in terms of the number of parts of solvent required to dissolve one part of solvent. A compound (solute) that is Very Soluble is one that requires less than one part of solvent per part of solute. A Freely Soluble compound requires from 1 to 10 parts of solvent per part of solute, and a soluble compound requires from 10 to 30 parts of solvent per part of solute. A Sparingly Soluble compound requires from 30 to 100 parts of solvent per part of solute. A Slightly Soluble compound requires from 100 to 1000 parts of solvent per part of solute. A Very Slightly Soluble compound requires from 1000 to 10,000 parts of solvent per part of solute, and a Practically Insoluble or Insoluble compound requires greater than or equal to 10,000 parts of solvent per part of solute. Such terms as used herein have the same meanings.

As used herein with reference made to antibiotics, drugs, pharmaceutical agents, or compounds, the terms “poorly-soluble” or “poorly-water soluble” shall mean that such antibiotic, drug, pharmaceutical agent, or compound is either Sparingly Soluble, Slightly Soluble, Very Slightly Soluble, Practically Insoluble or Insoluble as those terms are defined above.

As used herein, the term “macrolide antibiotic” refers to natural, synthetic, and semi-synthetic macrolide antibiotics. Exemplary macrolide antibiotics include but are not limited to azithromycin, clarithromycin, dirithromycin, erythromycin, oleandomycin, roxithromycin, troleandomycin, and derivatives thereof.

The non-lipophilic, non-polymeric excipient is water soluble and/or water erodible when present in a compressed solid dosage form or composition. Suitable exemplary excipients include non-lipophilic non-polymeric excipients selected from the group consisting of lactose, maltose, dextrose, sucrose, fructose, non-polymeric carbohydrate, alpha-hydroxy acid, polyols (such as mannitol, sorbitol, xylitol, and the like), dicalcium phosphate, calcium sulfate or a combination thereof. Exemplary saccharides include lactose, lactitol, glucose, sorbitol, or derivatives thereof. In one embodiment, the non-lipophilic, non-polymeric excipient is lactose.

The composition comprises an amphiphilic surfactant having a hydrophilic/lipophilic balance (HLB) of at least 10. In one embodiment, the surfactant has an HLB greater than 14. According to another embodiment, the surfactant has an HLB of about 40. Exemplary surfactants include, by way of example and without limitation, sodium lauryl sulfate, aerosol OT, cholic acid, a salt of cholic acid, or the like.

The amount of surfactant generally varies from 0.1% to 10% by weight of the composition. Most preferably the amount of surfactant varies from 0.75% to 5%.

As known to those skilled in the art surfactants may be combined to achieve a mixture having a desired HLB. Accordingly, the composition of the invention can also include a mixture of surfactants such that the HLB of the mixture is at least 10, at least 14, or at least 40. When a mixture of surfactants is used, the HLB value of the mixture can be calculated as follows:
(HLB_{A})×(FR_{A})+(HLB_{B})×(FR_{B})=HLB_{A+B}

wherein:

HLB_{A} denotes the HLB of surfactant A;

FR_{A} denotes the fraction (% wt.) of surfactant A in the mixture;

(HLB_{B}) denotes the HLB of surfactant B;

FR_{B} denotes the fraction (% wt.) of surfactant B in the mixture; and

HLB_{A+B} denotes the HLB of the mixture.

The composition may optionally contain bile salts and bile acids of cholic acid, taurocholic acid, deoxycholic acid, and others known to the artisan of pharmaceutics. The amount of bile salts and/or bile acids generally varies from 0% to about 8% by weight of the composition. Bile salts that may be used include, by way of example and without limitation, the sodium, potassium, calcium, and magnesium salts and alkaline salts of bile acids. Additionally, the bile acid or the bile salt may be intergranular or intragranular in the compressed dosage form.

Although extended release properties are achieved in accordance with the invention by using the extended release agents as described herein, whereby it is not necessary to add a release rate-retarding polymer to the composition, it is possible to include such a polymer in the composition. The only requirement is that the composition or formulation of the invention comprises a sufficient amount of surfactant and non-lipophilic non-polymeric excipient such that together they prolong the release of the macrolide antibiotic as compared to a substantially similar formulation excluding one or both of the surfactant and non-lipophilic, non-polymeric excipient.

The amount of macrolide antibiotic varies from about 30% to 80% by weight of the composition. In one embodiment, the composition comprises about 40% to 65% by weight of macrolide antibiotic. Generally, the solid oral dosage forms of the present invention contain clarithromycin in a dosage range of about 100 mg to about 1,000 mg. In specific embodiments, the solid oral dosage forms contain about 500 mg clarithromycin and from 250 mg to 600 mg of lactose with 1% to 3% sodium lauryl sulfate based upon the final composition.

Pharmaceutically acceptable non-lipophilic, non-polymeric excipients useful in the present invention include organic or inorganic compounds. Such excipients include, by way of example and without limitation, lactose, maltose, sucrose, dextrose, fructose, non-polymeric carbohydrates, alpha-hydroxy acids, inorganic electrolytes, mannitol, xylitol, sorbitol, sodium chloride, calcium sulfate, calcium phosphate, calcium carbonate, dicalcium phosphate, calcium sulfate, and others known to artisans in the field of pharmaceutics.

The amount of non-lipophilic, non-polymeric excipient typically ranges from about 20% to 80% by weight of the composition. In specific embodiments, the amount of non-lipophilic, non-polymeric excipient ranges from about 20-80% wt. of the composition.

In one embodiment, the composition comprises: a macrolide antibiotic present in an amount of about 40% to about 60% wt. of the composition; a surfactant, or a mixture of surfactants together, having an HLB value of at least 10 and being present in an amount of about 1% to about 3% wt. of the composition; lactose present in an amount of about 20% to about 60% by weight of the composition; and optionally one or more excipients, wherein the composition is essentially free of a polymer, wax, fat, fatty acid, and/or fatty alcohol.

The compressed composition of the invention can be made by various tableting processes known to those skilled in the art. For example, the composition is made by way of a granulation process wherein granules could be produced by wet, dry, melt, extrusion, or other processes as known in the art. In the granular compositions, the drug is included within the granules. In one embodiment, the drug is granulated in the absence of the non-lipophilic, non-polymeric excipient, and in another embodiment the drug is granulated in the presence of the non-lipophilic, non-polymeric excipient. The drug-containing granules are then mixed with an excipient composition and compressed to form the compressed composition. When the non-lipophilic, non-polymeric excipient is absent from the drug-containing granules but still present in the compressed composition, the non-lipophilic, non-polymeric excipient is mixed with the granules and then compressed to form the composition. In such a case, the non-lipophilic, non-polymeric excipient is “extragranular,” meaning that it occurs outside of the granulated composition that contains drug. When the non-lipophilic, non-polymeric excipient is present within the drug-containing granules it is said to be “intragranular.” However, the drug is typically granulated in the presence of the surfactant. The surfactant may be included “intragranularly” meaning that it is included in the drug-containing granules used to make the compressed composition and/or be may added extragranularly.

The composition and dosage form of the invention can also comprise an adsorbent, alkalizing agent, acidifying agent, antioxidant, buffering agent, colorant, flavorant, antiadherent, binder, diluent, direct compression excipient, disintegrant, glidant, lubricant, opaquant, plasticizer and/or polishing agent.

As used herein, the term “alkalizing agent” is intended to mean a compound used to provide alkaline medium. Such compounds include, by way of example and without limitation, ammonia solution, ammonium carbonate, diethanolamine, monoethanolamine, potassium hydroxide, sodium borate, sodium carbonate, sodium bicarbonate, sodium hydroxide, triethanolamine, diethanolamine, organic amine base, alkaline amino acids, trolamine, and others known to those of ordinary skill in the art.

As used herein, the term “acidifying agent” is intended to mean a compound used to provide an acidic medium. Such compounds include, by way of example and without limitation, acetic acid, acidic amino acids, citric acid, fumaric acid and other alpha hydroxy acids, hydrochloric acid, ascorbic acid, phosphoric acid, sulfuric acid, tartaric acid, nitric acid, and others known to those of ordinary skill in the art.

As used herein, the term “adsorbent” is intended to mean an agent capable of holding other molecules onto its surface by physical or chemical (chemisorption) means. Such compounds include, by way of example and without limitation, powdered and activated charcoal, and other materials known to one of ordinary skill in the art.

As used herein, the term “antioxidant” is intended to mean an agent that inhibits oxidation and thus is used to prevent the deterioration of preparations by the oxidative process. Such compounds include, by way of example and without limitation, ascorbic acid, ascorbyl palmitate, butylated hydroxyanisole, butylated hydroxytoluene, hypophophorous acid, monothioglycerol, propyl gallate, sodium ascorbate, vitamin E, vitamin E succinate, vitamin E derivatives, sodium bisulfite, sodium formaldehyde sulfoxylate, citric acid, vitamin A, vitamin A derivatives, EDTA, sodium metabisulfite, and other materials known to one of ordinary skill in the art.

As used herein, the term “buffering agent” is intended to mean a compound used to resist change in pH upon dilution or addition of acid or alkali. Such compounds include, by way of example and without limitation, potassium metaphosphate, potassium phosphate, monobasic sodium acetate, anhydrous and dihydrate sodium citrate, and other buffering materials known to one of ordinary skill in the art.

As used herein, the term “antiadherent” is intended to mean an agent that prevents the sticking of tablet formulation ingredients to punches and dies in a tableting machine during production. Such compounds include, by way of example and without limitation, magnesium stearate, talc, calcium stearate, zinc stearate, hydrogenated vegetable oil, mineral oil, stearic acid, silicon dioxide, and other materials known to one of ordinary skill in the art.

As used herein, the term “diluent” or “filler” is intended to mean an inert substance used as filler to create the desired bulk, flow properties, and compression characteristics in the preparation of tablets and capsules. Such compounds include, by way of example and without limitation, dibasic calcium phosphate, kaolin, lactose, sucrose, mannitol, precipitated calcium carbonate, sorbitol, and other materials known to one of ordinary skill in the art.

As used herein, the term “direct compression excipient” is intended to mean a compound used in direct compression tablet formulations. Such compounds include, by way of example and without limitation dibasic calcium phosphate, lactose, sucrose, mannitol, precipitated calcium carbonate, sorbitol, and other materials known to one of ordinary skill in the art.

As used herein, the term “glidant” is intended to mean agents used in tablet and capsule formulations to promote the flowability of a granulation. The amount of glidant generally varies from about 0.01% to about 5% by weight of the composition. Such compounds include, by way of example and without limitation, colloidal silicon dioxide, talc, calcium silicate, magnesium silicate, colloidal silicon, silicon hydrogel, tricalcium phosphate, and other materials known to one of ordinary skill in the art.

As used herein, the term “lubricant” is intended to mean substances used in tablet formulations to reduce friction during tablet compression. The amount of lubricant generally varies from about 0.01% to about 5% by weight of the composition. Such compounds include, by way of example and without limitation, calcium stearate, magnesium stearate, stearic acid, zinc stearate, sodium stearyl fumarate, adipic acid, benzoic acid, leucine, and other materials known to one of ordinary skill in the art and mixtures thereof.

As used herein, the term “opaquant” is intended to mean a compound used to render a capsule or a tablet coating opaque. Opaquants may be used alone or in combination with a colorant. Opaquant compounds include, by way of example and without limitation, titanium dioxide and other materials known to one of ordinary skill in the art.

As used herein, the term “colorant” is intended to mean a compound used to impart color to solid (e.g., tablets) pharmaceutical preparations. Such compounds include, by way of example and without limitation, FD&C Red No. 3, FD&C Red No. 20, FD&C Yellow No. 6, FD&C Blue No. 2, D&C Green No. 5, D&C Orange No. 5, D&C Red No. 8, caramel, and ferric oxide, red, other F.D. & C. dyes and natural coloring agents such as grape skin extract, beet red powder, beta-carotene, annato, carmine, turmeric, paprika, and other materials known to one of ordinary skill in the art. The amount of coloring agent used will vary as desired.

The composition may optionally contain wetting agents. Exemplary wetting agents include, by way of example and without limitation, glycerin, propylene glycol, propylene carbonate, others known to the artisan in the field of pharmaceutics, or mixtures thereof. The amount of wetting agent used generally varies from 0.10% to 10% by weight of the composition.

It should be understood, that compounds used in the art of pharmaceutical formulation generally serve a variety of functions or purposes. Thus, if a compound named herein is mentioned only once or is used to define more than one term herein, its purpose or function should not be construed as being limited solely to that named purpose(s) or function(s).

The compressed dosage forms are prepared by techniques known in the art and contain a therapeutically useful amount of clarithromycin and other such excipients as are necessary to form the compressed dosage form by such techniques. The final compositions are essentially free of polymers, waxes, fats, fatty acids, and/or fatty alcohols. The compressed dosage forms may be over-encapsulated by inclusion in gelatin capsules or shells.

A compressed composition or dosage form can be made according to the processes detailed in the examples below. In the most general sense, the macrolide antibiotic, surfactant and optionally the non-polymeric, non-lipophilic excipient are granulated to form a granular composition. The granular composition is then compressed optionally in the presence of other excipients. If the non-polymeric, non-lipophilic excipient of the invention was not included within the granules, then the granular composition is compressed at least in the presence of the non-polymeric, non-lipophilic excipient.

Granulation is effected by the addition of a liquid binder medium to a powdered solid comprising the macrolide antibiotic, surfactant and optionally non-polymeric, non-lipophilic excipient. The binder medium is preferably aqueous and may optionally contain wetting agents, bile acids, bile salts, pH modifiers, and/or binder materials or mixtures thereof. Other binder media are known to those skilled in the art. The liquid is dried from the granulation mixture thereby forming dried granules, the macrolide-containing granulation. In general, the particles of the macrolide-containing granulation range in size from about 10 microns to about 1000 microns in length or diameter. Preferrably the particles of the macrolide containing granulation range in size from about 150 microns to about 500 microns. The dried granules are optionally sieved and/or ground to the appropriate size. The macrolide-containing granulation is optionally blended with a binding composition comprising filler excipient, surfactant, wetting agent, bile acid, bile salt, glidant, lubricant, pH modifiers, colorants, flavorants, or mixtures thereof. The final mixture is then compressed into individual tablets or pills.

The formulations may also generally be prepared by dry blending the macrolide with the non-lipophilic, non-polymeric filler excipient followed by granulating the mixture using a solution or dispersion containing, a surfactant and/or a pH buffer, and optionally containing a carbohydrate such as sucrose to form a wet mass that is granulated. The granulation is conducted by methods known in the art. The wet granules are dried in an oven or fluid bed dryer, sieved, and/or ground to the appropriate size. A binding composition comprising additional excipient along with glidants and/or lubricants are mixed with the dried granulation to obtain the final granulation.

The compositions of the invention can be administered orally in the form of compressed dosage forms such as tablets or pills, wherein tablets or pills are individual dosage units typically prepared through the mechanical compression of an aliquot of powder and/or agglomerates or mixtures thereof. The tablets or pills can be shaped as desired using conventional tablet press equipment.

Compressed dosage forms can be prepared by techniques known in the art so that the compositions contain a therapeutically useful amount of macrolide antibiotic and such non-lipophilic, non-polymeric excipients as are necessary to form the compressed dosage form by such techniques. Other techniques for granulation that do not require lipophilic or polymeric excipients are known to those skilled in the art and may be used to affect particle agglomeration.

In the compressed dosage form, the excipients may be intergranular or intragranular. Where the excipients are intergranular, they are present in an amount of up to about 60% of the total compressed dosage form weight.

In general, the compressed dosage forms of the present invention are compressed so that they have a hardness of between 5-40 Kp. In general, the release rate of drug from the compressed composition can be reduced by increasing the hardness of the composition and vice versa.

The extended release characteristics of the compositions are assessed using in vitro dissolution assays generally performed according to Example 20. In one embodiment, the compositions of the present invention have a dissolution profile whereby, in 900 mL of 0.1 M sodium acetate buffer (pH 5.0) using U.S.P. Method II at 75 RPM, the composition releases 0% 5 to 45% clarithromycin after 2 hours, 10% to 65% after 4 hours, 40% to 85% after 6 hours and not less 70% clarithromycin after 10 hours.

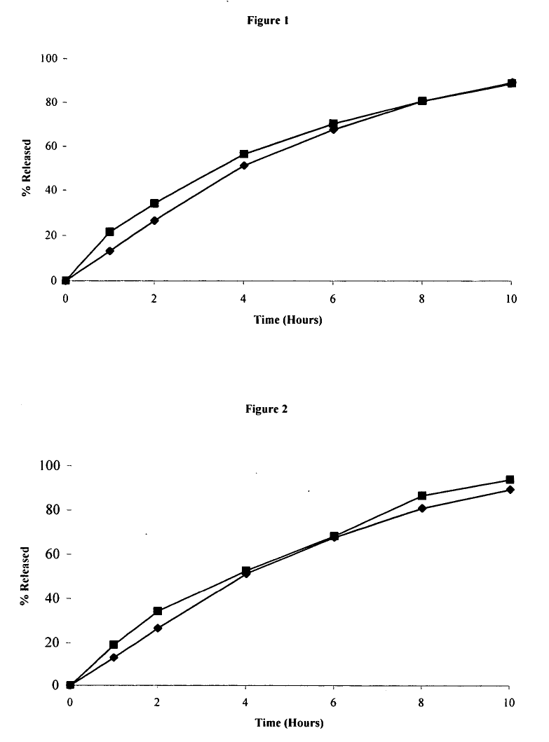

FIGS. 1-13 depict the release profiles observed for tablet dosage forms prepared according to Examples 1-13 respectively. In general, the macrolide antibiotic is released according to a first order, substantially first order, or sigmoidal release profile. The drug is released over an extended period of time, generally at least about 12, 16, 18, 20, or 24 hours after exposure to an aqueous environment of use, e.g. after oral administration to a subject or after exposure to an assay solution. The composition of the invention is administered to a host in an amount effective to treat a bacterial infection.

In conformity with the instant invention a unit dose of a macrolide antibiotic may be administered on a once or twice daily basis. In general, one or two tablets or pills are administered orally to a subject once or twice daily. For example, a single tablet may be administered once or twice daily, or two tablets may be administered once or twice daily. Each tablet can contain a unit dose or half of a unit dose. Dosing regimens utilizing the instant invention for a particular subject would be decided clinically by a prescribing physician. The subject-specific dosing regimen would of course depend upon the severity of the disorder being treated, the overall health of the subject, the weight and/or age of the subject, the dose strength of the tablet(s) prescribed, the macrolide release profile of the tablet prescribed, and be further informed by other considerations known in the bacterial infection arts.

The invention provides a method of treating a bacterial infection in a subject comprising the step of administering to the subject a compressed composition or dosage form as described herein. The Physician's Desk Reference 56^{th} ed. (2002) sets forth the recommended dosages for adults and children according to the bacterial infection being treated with clarithromycin or erythromycin. Although, the macrolide antibiotics included within the scope of the invention vary in structure and therapeutic activity, the following guidelines are useful for initiating therapy with a macrolide antibiotic in a human. In general, 250-500 mg of the macrolide are administered twice daily for a period of 7-14 days. Such a dosing regimen is useful for treating a wide range of bacterial infections in humans. For children, the dose is determined according to body weight. In general, a dose of 7.5-25 mg of macrolide per kg of body weight is administered on a twice daily basis. Generally, the total dose of macrolide should not exceed 4 grams per day.

The compressed composition or dosage form can be used to treat bacterial infections such as pharyngitis, tonsillitis, acute maxillary sinusitis, acute exacerbation of chronic bronchitis, pneumonia, uncomplicated skin and skin structure infections, disseminated mycobacterial infections, acute pelvic inflammatory disease, intestinal amebiasis, erythrasma, diphtheria, pertussis, respiratory (upper and lower) tract infections, listeriosis, gram-negative bacterial infection, gram-positive bacterial infection, and other microbial infections.

The term “unit dosage form” is used herein to mean a single or multiple dose form containing a quantity of the active ingredient, and the excipients and/or carriers, said quantity being such that one or more predetermined units are normally required for a single therapeutic administration. It will be understood that the specific dose level that may be employed with the instant invention for any patient will be discernible to one of ordinary skill in the art taking into consideration such art recognized factors as the indication being treated, the therapeutic agent employed, the activity of therapeutic agent, the severity of the indication, the patient's health, the patient's age, the patient's sex, the patient's weight, the patient's diet, the patient's pharmacological response, the specific dosage form employed, and other considerations known in the bacterial infections art.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “patient” is taken to mean warm blooded animals such as mammals, for example, cats, dogs, mice, guinea pigs, horses, bovine cows, sheep, and humans.

The formulation of the invention will comprise an effective amount of macrolide antibiotic. By the term “effective amount”, it is understood that a therapeutically effective amount is contemplated. A therapeutically effective amount is the amount or quantity of clarithromycin that is sufficient to elicit the required or desired therapeutic response, or in other words, the amount that is sufficient to elicit an appreciable biological response when administered to a subject. In practicing the instant invention, one of ordinary skill in the art would readily discern the effective amount of macrolide antibiotic by taking into consideration its intended function in treating known bacterial infections such as the non-limiting examples mentioned hererinabove and hereinbelow.

In view of the above description and the examples below, one of ordinary skill in the art will be able to practice the invention as claimed without undue experimentation. The foregoing will be better understood with reference to the following examples that detail certain procedures for the preparation of formulations according to the present invention. All references made to these examples are for the purposes of illustration. The following examples should not be considered exhaustive, but merely illustrative of only a few of the many embodiments contemplated by the present invention.

In the following examples, the reference product is Biaxin® XL Filmtab® (clarithromycin extended-release tablets, Abbott) purchased through commercial pharmaceutical distribution channels.

### EXAMPLE 1

A compressed extended release tablet containing clarithromycin was prepared according to the following procedure. The ingredients and the amount used are detailed in the table below. A macrolide and a portion of non-polymeric, non-lipophilic excipient are granulated with an aqueous buffer solution containing surfactant thereby forming a granulate comprising all three components. The granules are then mixed with the remaining non-polymeric, non-lipophilic excipient and other excipients and compressed into individual tablets. Accordingly, the macrolide, non-polymeric, non-lipophilic excipient and surfactant are all intragranular.

| Ingredient | Mg/Tablet |
|---|---|
| Clarithromycin | 500.0 |
| Lactose monohydrate, spray-dried | 554.0 |
| Colloidal silicon dioxide | 5.0 |
| Magnesium stearate | 5.0 |
| Sodium Lauryl Sulfate | 1.8 |
| Sodium Acetate Trihydrate | 0.2 |
| Total | 1066.0 |

The clarithromycin was dry blended with 90% of the lactose. The mixture was granulated with an aqueous dispersion of 1% sodium lauryl sulfate in 10 mM acetate buffer pH 5 until proper granulation was obtained. The granulation was dried and sieved.

The remaining lactose, colloidal silicon dioxide, and magnesium stearate were sieved and blended with the dry granulation to obtain a final blend. The final blend was compressed into tablets.

The tablets were evaluated in vitro in 1000 mL of 25 mM phosphate buffer/50 mM acetate buffer at pH 4.5 in a USP dissolution apparatus 2 at 75 RPM. The dissolution profile of clarithromycin from tablets prepared according to Example 1 is provided in FIG. 1.

### EXAMPLE 2

A compressed extended release tablet containing clarithromycin was prepared according to the following procedure. The ingredients and the amount used are detailed in the table below. The procedure of Example 1 was used with some slight modifications.

| Ingredient | Mg/Tablet |
|---|---|
| Clarithromycin | 500.0 |
| Lactose monohydrate, spray-dried | 290.0 |
| Colloidal silicon dioxide | 4.4 |
| Magnesium stearate | 4.4 |
| Sodium Lauryl Sulfate | 1.9 |
| Monobasic Potassium Phosphate | 1.1 |
| Total | 801.8 |

The clarithromycin was dry blended with 86% of the lactose. The mixture was granulated with an aqueous dispersion of 1% sodium lauryl sulfate in 50 mM phosphate buffer pH 5 until proper granulation was obtained. The granulation was dried, sieved, and sized.

The remaining lactose, colloidal silicon dioxide, and magnesium stearate were sieved and blended with the dry granulation to obtain a final blend. The final blend was compressed into tablets.

The tablets were evaluated in vitro in 1000 mL of 25 mM phosphate buffer/50 mM acetate buffer at pH 4.5 in a USP dissolution apparatus 2 at 75 RPM. The dissolution profile of clarithromycin from tablets prepared according to Example 2 is detailed in FIG. 2.

### EXAMPLE 3

A compressed extended release tablet containing clarithromycin was prepared according to the following procedure. The ingredients and the amount used are detailed in the table below. A macrolide was granulated with an aqueous buffer solution containing surfactant thereby forming a granulate comprising the two components. The granules were then mixed with the non-polymeric, non-lipophilic excipient(s) and other excipients and compressed into individual tablets. Accordingly, the macrolide and surfactant are all intragranular and the non-polymeric, non-lipophilic excipient is intergranular.

| Ingredient | Mg/Tablet |
|---|---|
| Clarithromycin | 500.0 |
| Lactose monohydrate, spray-dried | 283.0 |
| Dibasic Calcium Phosphate Anhydrous | 94.3 |
| Colloidal silicon dioxide | 4.0 |
| Magnesium stearate | 4.0 |
| Sodium Lauryl Sulfate | 1.8 |
| Monobasic Potassium Phosphate | 1.2 |
| Total | 888.3 |

The clarithromycin was granulated with an aqueous dispersion of 1% sodium lauryl sulfate in 50 mM phosphate buffer pH 5 until proper granulation was obtained. The granulation was dried, sieved, and sized.

The lactose, dibasic calcium phosphate anhydrous, colloidal silicon dioxide, and magnesium stearate were sieved and blended with the dry granulation to obtain a final blend. The final blend was compressed into tablets.

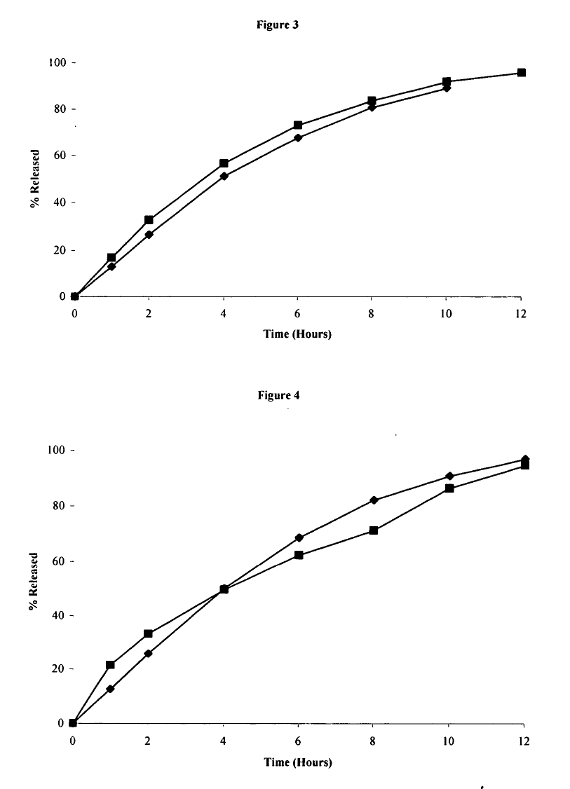

The tablets were evaluated in vitro in 1000 mL of 25 mM phosphate buffer/50 mM acetate buffer at pH 4.5 in a USP dissolution apparatus 2 at 75 RPM. The dissolution profile of clarithromycin from tablets prepared according to Example 3 is detailed in FIG. 3.

### EXAMPLE 4

A compressed extended release tablet containing clarithromycin was prepared according to the following procedure. The ingredients and the amount used are detailed in the table below. These tablets were made according to the procedure of Example 1 with some modifications.

| Ingredient | mg/Tablet |
|---|---|
| Clarithromycin | 500.0 |
| Lactose monohydrate, spray-dried | 290.0 |
| Potassium Phosphate | 1.4 |
| Sodium Lauryl Sulfate | 1.6 |
| Colloidal silicon dioxide | 4.4 |
| Magnesium stearate | 4.4 |
| Total | 801.8 |

The clarithromycin was dry blended with 86.2% of the lactose. The mixture was granulated with an aqueous dispersion of 1% sodium lauryl sulfate in 50 mM phosphate buffer pH 5 until proper granulation was obtained. The granulation was dried, sieved, and sized.

The remaining lactose, colloidal silicon dioxide, and magnesium stearate were sieved and blended with the dry granulation to obtain a final blend. The final blend was compressed into tablets.

The tablets were evaluated in vitro in 900 mL of 100 mM acetate buffer at pH 5 in a USP dissolution apparatus 2 at 75 RPM. The dissolution profile of clarithromycin from tablets prepared according to Example 4 is detailed in FIG. 4.

### EXAMPLE 5

A compressed extended release tablet containing clarithromycin was prepared according to the following procedure. The ingredients and the amount used are detailed in the table below. These tablets were made according to the procedure of Example 3 with some modifications.

| Ingredient | Mg/Tablet |
|---|---|
| Clarithromycin | 500.0 |
| Lactose monohydrate, spray-dried | 414.3 |
| Potassium Phosphate | 1.2 |
| Sodium Lauryl Sulfate | 1.8 |
| Colloidal silicon dioxide | 5.0 |
| Magnesium stearate | 5.0 |
| Total | 927.3 |

The clarithromycin was granulated with an aqueous dispersion of 1% sodium lauryl sulfate in 50 mM phosphate buffer pH 5 until proper granulation was obtained. The granulation was dried, sieved, and sized.

The lactose, colloidal silicon dioxide, and magnesium stearate were sieved and blended with the dry granulation to obtain a final blend. The final blend was compressed into tablets.

The tablets were evaluated in vitro in 900 mL of 100 mM acetate buffer at pH 5 in a USP dissolution apparatus 2 at 75 RPM. The dissolution profile of clarithromycin from tablets prepared according to Example 5 is detailed in FIG. 5.

### EXAMPLE 6

A compressed extended release tablet containing clarithromycin was prepared according to the following procedure. The ingredients and the amount used are detailed in the table below. These tablets were made according to the procedure of Example 3 with some modifications.

| Ingredient | Mg/Tablet |
|---|---|
| Clarithromycin | 500.0 |
| Lactose monohydrate, spray-dried | 451.3 |
| Potassium Phosphate | 1.2 |
| Sodium Lauryl Sulfate | 1.8 |
| Colloidal silicon dioxide | 5.0 |
| Magnesium stearate | 5.0 |
| Total | 964.3 |

The clarithromycin was granulated with an aqueous dispersion of 1% sodium lauryl sulfate in 50 mM phosphate buffer pH 5 until proper granulation was obtained. The granulation was dried, sieved, and sized.

The lactose, colloidal silicon dioxide, and magnesium stearate were sieved and blended with the dry granulation to obtain a final blend. The final blend was compressed into tablets.

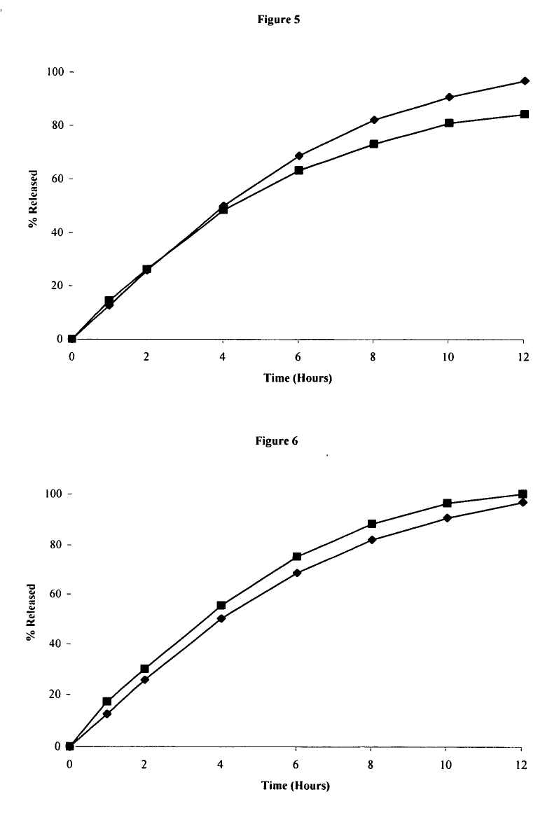

The tablets were evaluated in vitro in 900 mL of 100 mM acetate buffer at pH 5 in a USP dissolution apparatus 2 at 75 RPM. The dissolution profile of clarithromycin from tablets prepared according to Example 6 is detailed in FIG. 6.

### EXAMPLE 7

A compressed extended release tablet containing clarithromycin was prepared according to the following procedure. The ingredients and the amount used are detailed in the table below. A macrolide and a first non-polymeric, non-lipophilic excipient are granulated with an aqueous buffer solution containing surfactant, thereby forming a granulate comprising all three components. The granules are then mixed with a different second non-polymeric, non-lipophilic excipient and other excipients and compressed into individual tablets. Accordingly, the macrolide, first non-polymeric, non-lipophilic excipient and surfactant are all intragranular and the second non-polymeric, non-lipophilic excipient is intergranular.

| Ingredient | mg/Tablet |
|---|---|
| Clarithromycin | 510.3 |
| Lactose monohydrate, spray-dried | 295.7 |
| Sodium Lauryl Sulfate | 1.4 |
| Maltose | 40.5 |
| Colloidal silicon dioxide | 4.3 |
| Magnesium stearate | 4.3 |
| Total | 856.5 |

The clarithromycin was dry blended with the lactose. The mixture was granulated with an aqueous solution of 1% sodium lauryl sulfate until proper granulation was obtained. The granulation was dried, sieved, and sized.

The maltose, colloidal silicon dioxide, and magnesium stearate were sieved and blended with the dry granulation to obtain a final blend. The final blend was compressed into tablets.

The tablets were evaluated in vitro in 900 mL of 100 mM acetate buffer at pH 5 in a USP dissolution apparatus 2 at 75 RPM. The dissolution profile of clarithromycin from tablets prepared according to Example 7 is detailed in FIG. 7.

### EXAMPLE 8

A compressed extended release tablet containing clarithromycin was prepared according to the following procedure. The ingredients and the amount used are detailed in the table below. These tablets were made according to the procedure of Example 7 with some modifications.

| Ingredient | Mg/Tablet |
|---|---|
| Clarithromycin | 501.2 |
| Sorbitol | 250.6 |
| Lactose monohydrate, spray-dried | 40.0 |
| Sodium Lauryl Sulfate | 1.2 |
| Colloidal silicon dioxide | 4.4 |
| Magnesium stearate | 4.4 |
| Total | 801.8 |

The clarithromycin was dry blended with the sorbitol. The mixture was granulated with an aqueous solution of 1% sodium lauryl sulfate until proper granulation was obtained. The granulation was dried, sieved, and sized.

The lactose, colloidal silicon dioxide, and magnesium stearate were sieved and blended with the dry granulation to obtain a final blend. The final blend was compressed into tablets.

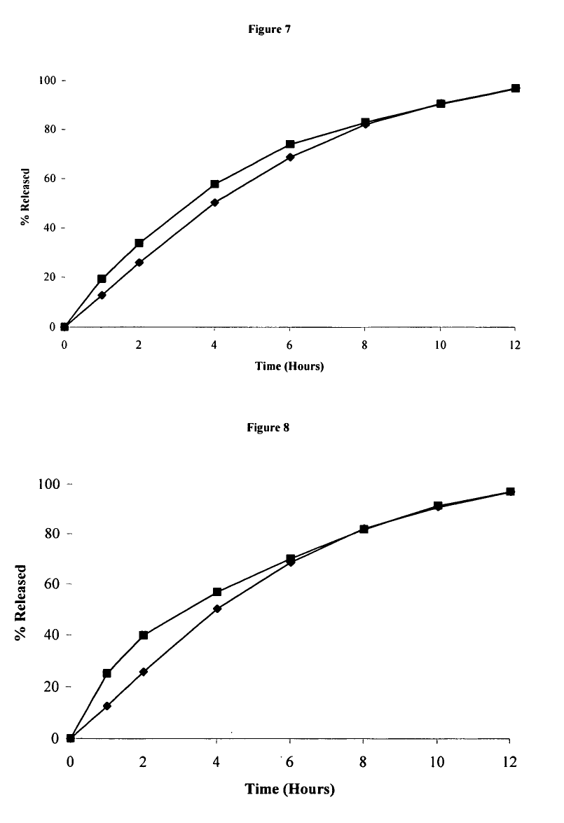

The tablets were evaluated in vitro in 900 mL of 100 mM acetate buffer at pH 5 in a USP dissolution apparatus 2 at 75 RPM. The dissolution profile of clarithromycin from tablets prepared according to Example 8 is detailed FIG. 8.

### EXAMPLE 9

A compressed extended release tablet containing clarithromycin was prepared according to the following procedure. The ingredients and the amount used are detailed in the table below. A macrolide and two different non-polymeric, non-lipophilic excipients are granulated with an aqueous buffer solution containing surfactant thereby forming a granulate comprising all four components. The granules are then mixed with the remaining portion of one of the non-polymeric, non-lipophilic excipients and other excipients and compressed into individual tablets. Accordingly, the macrolide, two different non-polymeric, non-lipophilic excipients and the surfactant are all intragranular, and one of the non-polymeric, non-lipophilic excipients is also intergranular.

| Ingredient | mg/Tablet |
|---|---|
| Clarithromycin | 494.0 |
| Sorbitol | 24.3 |
| Lactose monohydrate, spray-dried | 287.8 |
| Sodium Lauryl Sulfate | 1.6 |
| Colloidal silicon dioxide | 4.1 |
| Magnesium stearate | 4.1 |
| Total | 815.9 |

The clarithromycin was dry blended with the 87% of the lactose. The mixture was granulated with an aqueous solution of 1% sodium lauryl sulfate and 15% sorbitol until proper granulation was obtained. The granulation was dried, sieved, and sized.

The remaining lactose, colloidal silicon dioxide, and magnesium stearate were sieved and blended with the dry granulation to obtain a final blend. The final blend was compressed into tablets.

The tablets were evaluated in vitro in 900 mL of 100 mM acetate buffer at pH 5 in a USP dissolution apparatus 2 at 75 RPM. The dissolution profile of clarithromycin from tablets prepared according to Example 9 is detailed in FIG. 9.

### EXAMPLE 10

A compressed extended release tablet containing clarithromycin was prepared according to the following procedure. The ingredients and the amount used are detailed in the table below. A macrolide and a non-polymeric, non-lipophilic excipient are granulated with an aqueous buffer solution containing a first surfactant thereby forming a granulate comprising all three components. The granules are then mixed with the remaining of the first surfactant and a bile acid and other excipients and compressed into individual tablets. Accordingly, the macrolide, non-polymeric, non-lipophilic excipient and the first surfactant are all intragranular and the first surfactant and bile acid are intergranular.

| Ingredient | mg/Tablet |
|---|---|
| Clarithromycin | 510.5 |
| Sorbitol | 255.3 |
| Sodium Lauryl Sulfate | 8.9 |
| Sodium Deoxycholate | 16.8 |
| Colloidal silicon dioxide | 3.9 |
| Magnesium stearate | 3.9 |
| Total | 799.3 |

The clarithromycin was dry blended with the sorbitol. The mixture was granulated with an aqueous solution of 1% sodium lauryl sulfate until proper granulation was obtained. The granulation was dried, sieved, and sized.

The additional sodium lauryl sulfate, sodium deoxycholate, colloidal silicon dioxide, and magnesium stearate were sieved and blended with the dry granulation to obtain a final blend. The final blend was compressed into tablets.

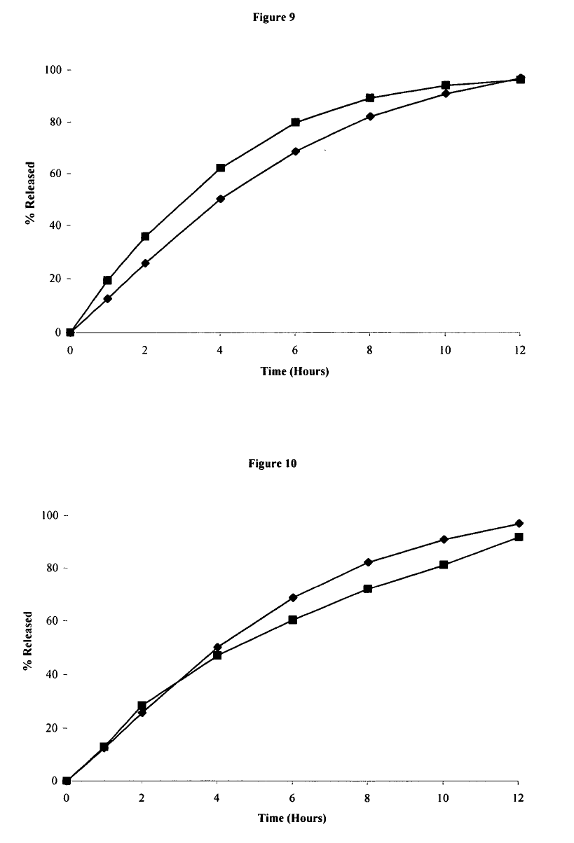

The tablets were evaluated in vitro in 900 mL of 100 mM acetate buffer at pH 5 in a USP dissolution apparatus 2 at 75 RPM. The dissolution profile of clarithromycin from tablets prepared according to Example 10 is detailed in FIG. 10.

### EXAMPLE 11

A compressed extended release tablet containing clarithromycin was prepared according to the following procedure. The ingredients and the amount used are detailed in the table below. A macrolide and a non-polymeric, non-lipophilic excipient are granulated with an aqueous buffer solution containing a bile acid, thereby forming a granulate comprising all three components. The granules are then mixed with a surfactant and the remaining portion of bile acid and other excipients and compressed into individual tablets. Accordingly, the macrolide, non-polymeric, non-lipophilic excipient and the bile acid are all intragranular, and the surfactant and bile acid are intergranular.

| Ingredient | mg/Tablet |
|---|---|
| Clarithromycin | 510.5 |
| Sorbitol | 255.3 |
| Sodium Lauryl Sulfate | 7.8 |
| Sodium Deoxycholate | 5.0 |
| Colloidal silicon dioxide | 3.9 |
| Magnesium stearate | 3.9 |
| Total | 786.4 |

The clarithromycin was dry blended with the sorbitol. The mixture was granulated with an aqueous solution of 1% sodium deoxycholate until proper granulation was obtained. The granulation was dried, sieved, and sized.

The sodium lauryl sulfate, 79% of the sodium deoxycholate, colloidal silicon dioxide, and magnesium stearate were sieved and blended with the dry granulation to obtain a final blend. The final blend was compressed into tablets.

The tablets were evaluated in vitro in 900 mL of 100 mM acetate buffer at pH 5 in a USP dissolution apparatus 2 at 75 RPM. The dissolution profile of clarithromycin from tablets prepared according to Example 11 is detailed in FIG. 11.

### EXAMPLE 12

A compressed extended release tablet containing clarithromycin was prepared according to the following procedure. The ingredients and the amount used are detailed in the table below. The following tablets were made according to the procedure of Example 11 with some modifications.

| Ingredient | mg/Tablet |
|---|---|
| Clarithromycin | 510.5 |
| Sorbitol | 255.3 |
| Sodium Lauryl Sulfate | 7.8 |
| Sodium Deoxycholate | 7.0 |
| Colloidal silicon dioxide | 3.9 |
| Magnesium stearate | 3.9 |
| Total | 788.4 |

The clarithromycin was dry blended with the sorbitol. The mixture was granulated with an aqueous solution of 1% sodium deoxycholate until proper granulation was obtained. The granulation was dried, sieved, and sized.

The sodium lauryl sulfate, 84% of the sodium deoxycholate, colloidal silicon dioxide, and magnesium stearate were sieved and blended with the dry granulation to obtain a final blend. The final blend was compressed into tablets.

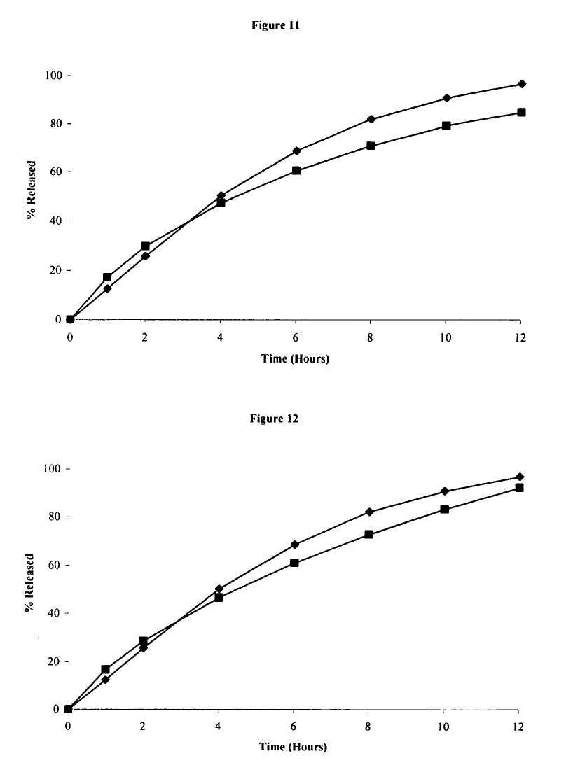

The tablets were evaluated in vitro in 900 mL of 100 mM acetate buffer at pH 5 in a USP dissolution apparatus 2 at 75 RPM. The dissolution profile of clarithromycin from tablets prepared according to Example 12 is detailed in FIG. 12.

### EXAMPLE 13

A compressed extended release tablet containing clarithromycin was prepared according to the following procedure. The ingredients and the amount used are detailed in the table below. A macrolide and a portion of a first non-polymeric, non-lipophilic excipients are granulated with an aqueous buffer solution containing surfactant and a second non-polymeric, non-lipophilic excipient thereby forming a granulate comprising all four components. The granules are then mixed with the remaining non-polymeric, non-lipophilic excipients and a bile acid, and with other excipients, and then compressed into individual tablets. Accordingly, the macrolide, two different non-polymeric, non-lipophilic excipients and surfactant are all intragranular and the surfactant and one of the non-polymeric, non-lipophilic excipients are intergranular.

| Ingredient | mg/Tablet |
|---|---|
| Clarithromycin | 493.9 |
| Sorbitol | 24.4 |
| Lactose monohydrate, spray-dried | 247.0 |
| Sodium Lauryl Sulfate | 1.6 |
| Sodium Deoxycholate | 2.1 |
| Colloidal silicon dioxide | 4.1 |
| Magnesium stearate | 4.1 |
| Total | 777.2 |

The clarithromycin was dry blended with the lactose. The mixture was granulated with an aqueous solution of 1% sodium lauryl sulfate/15% sorbitol until proper granulation was obtained. The granulation was dried, sieved, and sized.

The sodium deoxycholate, colloidal silicon dioxide, and magnesium stearate were sieved and blended with the dry granulation to obtain a final blend. The final blend was compressed into tablets.

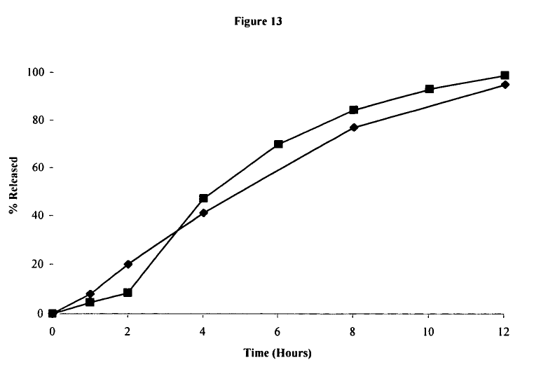

The tablets were evaluated in vitro in 900 mL of 0.03 M potassium phosphate/0.07 M sodium acetate buffer at pH 6 in a USP dissolution apparatus 2 at 75 RPM. The dissolution profile of clarithromycin from tablets prepared according to Example 13 is detailed in FIG. 13.

### EXAMPLE 14

A compressed extended release tablet containing clarithromycin was prepared according to the following procedure. The ingredients and the amount used are detailed in the table below. A macrolide and mixture of two different non-polymeric, non-lipophilic excipients are granulated with an aqueous buffer solution containing surfactant, thereby forming a granulate comprising all three components. The granules are then mixed with the remaining non-polymeric, non-lipophilic excipient and other excipients and compressed into individual tablets. Accordingly, the macrolide, non-polymeric, non-lipophilic excipients and surfactant are all intragranular.

| Ingredient | Mg/Tablet |
|---|---|
| Clarithromycin | 500.0 |
| Lactose monohydrate, spray-dried | 275.0 |
| Colloidal silicon dioxide | 4.3 |
| Magnesium stearate | 4.3 |
| Sodium Lauryl Sulfate | 5.0 |
| Sucrose | 86.6 |
| Total | 875.2 |

The clarithromycin was dry blended with 91% of the lactose. The mixture was granulated with an aqueous dispersion of 3.1% sodium lauryl sulfate in 49.5% sucrose until proper granulation was obtained. The granulation was dried, sieved, and sized.

The remaining lactose, colloidal silicon dioxide, and magnesium stearate were sieved and blended with the dry granulation to obtain a final blend. The final blend was compressed into tablets.

The tablets were evaluated in vitro in 900 mL of 100 mM acetate buffer at pH 5 in a USP dissolution apparatus 2 at 50 RPM.

### EXAMPLE 15

A compressed extended release tablet containing clarithromycin was prepared according to the following procedure. The ingredients and the amount used are detailed in the table below. These tablets were made according to the procedure of Example 14 with some modifications.

| Ingredient | Mg/Tablet |
|---|---|
| Clarithromycin | 500.0 |
| Lactose monohydrate, spray-dried | 297.2 |
| Dibasic Calcium Phosphate Dihydrate | 47.0 |
| Colloidal silicon dioxide | 5.0 |
| Magnesium stearate | 5.0 |
| Sodium Lauryl Sulfate | 5.0 |
| Sucrose | 86.7 |
| Total | 945.9 |

The clarithromycin was dry blended with 84% of the lactose. The mixture was granulated with an aqueous dispersion of 3.1% sodium lauryl sulfate in 49.5% sucrose until proper granulation was obtained. The granulation was dried, sieved, and sized.

The remaining lactose, dibasic calcium phosphate dihydrate, colloidal silicon dioxide, and magnesium stearate were sieved and blended with the dry granulation to obtain a final blend. The final blend was compressed into tablets.

The tablets were evaluated in vitro in 900 mL of 100 mM acetate buffer at pH 5 in a USP dissolution apparatus 2 at 50 RPM.

### EXAMPLE 16

A compressed extended release tablet containing clarithromycin was prepared according to the following procedure. The ingredients and the amount used are detailed in the table below. These tablets were made according to the procedure of Example 15 with some modifications.

| Ingredient | Mg/Tablet |
|---|---|
| Clarithromycin | 500.0 |
| Dibasic Calcium Phosphate Anhydrous | 333.6 |
| Lactose monohydrate, spray-dried | 49.6 |
| Colloidal silicon dioxide | 4.7 |
| Magnesium stearate | 4.7 |
| Sodium Lauryl Sulfate | 5.3 |
| Sucrose | 85.2 |
| Total | 983.1 |

The clarithromycin was dry blended with the dibasic calcium phosphate anhydrous. The mixture was granulated with an aqueous dispersion of 3.1% sodium lauryl sulfate in 49.5% sucrose until proper granulation was obtained. The granulation was dried, sieved, and sized.

The lactose, colloidal silicon dioxide, and magnesium stearate were sieved and blended with the dry granulation to obtain a final blend. The final blend was compressed into tablets.

The tablets were evaluated in vitro in 900 mL of 50 mM phosphate buffer at pH 4 in a USP dissolution apparatus 2 at 75 RPM.

### EXAMPLE 17

A compressed extended release tablet containing clarithromycin was prepared according to the following procedure. The ingredients and the amount used are detailed in the table below. These tablets were made according to the procedure of Example 3 with some modifications.

| Ingredient | mg/Tablet |
|---|---|
| Clarithromycin | 500.0 |
| Lactose monohydrate, spray-dried | 377.3 |
| Potassium Phosphate | 1.2 |
| Sodium Lauryl Sulfate | 1.8 |
| Colloidal silicon dioxide | 4.0 |
| Magnesium stearate | 4.0 |
| Total | 888.3 |

The clarithromycin was granulated with an aqueous dispersion of 1% sodium lauryl sulfate in 50 mM phosphate buffer pH 5 until proper granulation was obtained. The granulation was dried, sieved, and sized.

The lactose, colloidal silicon dioxide, and magnesium stearate were sieved and blended with the dry granulation to obtain a final blend. The final blend was compressed into tablets.

The tablets were evaluated in vitro in 900 mL of 100 mM acetate buffer at pH 5 in a USP dissolution apparatus 2 at 75 RPM.

### EXAMPLE 18

A compressed extended release tablet containing clarithromycin was prepared according to the following procedure. The ingredients and the amount used are detailed in the table below. A macrolide and a portion of a first non-polymeric, non-lipophilic excipients are granulated with an aqueous buffer solution containing surfactant and a second non-polymeric, non-lipophilic excipient, thereby forming a granulate comprising all four components. The granules are then mixed with the remaining non-polymeric, non-lipophilic excipients and surfactant and other excipients and compressed into individual tablets. Accordingly, the macrolide, two different non-polymeric, non-lipophilic excipients, and surfactant are all intragranular and the surfactant and one of the non-polymeric, non-lipophilic excipients are intergranular.

| Ingredient | mg/Tablet |
|---|---|
| Clarithromycin | 493.9 |
| Sorbitol | 24.4 |
| Lactose monohydrate, spray-dried | 288.1 |
| Sodium Lauryl Sulfate | 9.4 |
| Fumaric Acid | 16.8 |
| Colloidal silicon dioxide | 3.9 |
| Magnesium stearate | 3.9 |
| Total | 840.4 |

The clarithromycin was dry blended with 85% of the lactose. The mixture was granulated with an aqueous solution of 1% sodium lauryl sulfate/15% sorbitol until proper granulation was obtained. The granulation was dried, sieved, and sized.

The remaining sodium lauryl sulfate (83% of total), remaining lactose, colloidal silicon dioxide, fumaric acid, and magnesium stearate were sieved and blended with the dry granulation to obtain a final blend. The final blend was compressed into tablets.

The tablets were evaluated in vitro in 900 mL of 0.03 M potassium phosphate/0.07 M sodium acetate buffer at pH 6 in a USP dissolution apparatus 2 at 75 RPM.

### EXAMPLE 19

A compressed extended release tablet containing clarithromycin was prepared according to the following procedure. The ingredients and the amount used are detailed in the table below.

| Ingredient | mg/Tablet |
|---|---|
| Clarithromycin | 509.6 |
| Lactose monohydrate, spray-dried | 295.6 |
| Sodium Lauryl Sulfate | 7.8 |
| Sodium Deoxycholate | 2.5 |
| Colloidal silicon dioxide | 4.1 |
| Magnesium stearate | 4.1 |
| Total | 823.7 |

The clarithromycin was dry blended with 86% of the lactose. The mixture was granulated with an aqueous solution of 1% sodium deoxycholate until proper granulation was obtained. The granulation was dried, sieved, and sized.

The remaining lactose, sodium lauryl sulfate, colloidal silicon dioxide, and magnesium stearate were sieved and blended with the dry granulation to obtain a final blend. The final blend was compressed into tablets.

The tablets were evaluated in vitro in 900 mL of 0.03 M potassium phosphate/0.07 M sodium acetate buffer at pH 6 in a USP dissolution apparatus 2 at 75 RPM. The dissolution profile of clarithromycin from tablets prepared according to Example 19 is detailed in FIG. 19.

### EXAMPLE 20

The compressed composition is subjected to a dissolution assay as follows:

The pharmaceutical compositions are subjected to dissolution testing as described in the USP <711> dissolution monograph of the USP 27/NF 22 by using apparatus 2, which is equipped with rotating paddles and a vessel. The pharmaceutical compositions are evaluated in 900 mL of 100 mM sodium acetate buffer at pH 5. The pharmaceutical compositions may optionally be evaluated in 900 to 1000 mL of: 50 mM potassium phosphate buffer at pH 4, 25 mM potassium phosphate/50 mM sodium acetate buffer at pH 4.5, or 30 mM potassium phosphate/70 mM sodium acetate buffer at pH 6. The temperature of the medium is maintained at 37° C., and the paddles are rotated at 50 to 75 RPM. The quantity of clarithromycin in solution is determined from samples of the dissolution medium acquired at specific time intervals.

The above is a detailed description of particular embodiments of the invention. It will be appreciated that, although specific embodiments of the invention have been described herein for purposes of illustration, various modifications may be made without departing from the spirit and scope of the invention. Accordingly, the invention is not limited except as by the appended claims. All of the embodiments disclosed and claimed herein can be made and executed without undue experimentation in light of the present disclosure.

We claim:

1. A compressed extended release solid pharmaceutical composition comprising:
a) a macrolide antibiotic;
b) one or more surfactants;
c) one or more non-lipophilic, non-polymeric excipients; and
d) optionally one or more pharmaceutically acceptable excipients; wherein
the surfactant and the non-lipophilic, non-polymeric excipient cooperate to provide an extended release of macrolide antibiotic when the composition is exposed to an aqueous environment of use.

2. The composition of claim 1, wherein the pharmaceutical composition is made by mixing together and then compressing a granular composition with other functional excipients.

3. The composition of claim 2, wherein the granular composition comprises the macrolide antibiotic, one or more surfactants and optionally one or more non-lipophilic, non-polymeric excipients, and wherein the composition further comprises a binding composition.

4. The composition of claim 3, wherein at least one non-lipophilic, non-polymeric excipient is disposed within the granular composition.

5. The composition of claim 3, wherein at least one non-lipophilic, non-polymeric excipient is disposed within the binding composition.

6. The composition of claim 3, wherein a non-lipophilic, non-polymeric excipient is disposed within the granular composition and the binding composition.

7. The composition of claim 3, wherein the surfactant is disposed within the granular composition and the binding composition.

8. The composition of claim 2, wherein the surfactant is present in an amount of up to about 10% wt. of the composition.

9. The composition of claim 8, wherein the surfactant has an HLB value of at least 10.

10. The composition of claim 8, wherein the surfactant is selected from the group consisting of sodium lauryl sulfate, cholic acid, salts of cholic acid, Aerosil OT.

11. The composition of claim 2, wherein the macrolide antibiotic is present in an amount of about 30-80% wt. of the composition.

12. The composition of claim 11, wherein the macrolide antibiotic is selected from the group consisting of azithromycin, clarithromycin, dirithromycin, erythromycin, oleandomycin, roxithromycin, troleandomycin, or a derivative thereof.

13. The composition of claim 2, wherein the macrolide antibiotic is present in an amount of about 100-1000 mg.

14. The composition of claim 2, wherein the non-lipophilic non-polymeric excipient is present in an amount of about 20-60% wt. of the composition.

15. The composition of claim 14, wherein the non-lipophilic, non-polymeric excipient is a monosaccharide or disaccharide.

16. The composition of claim 14, wherein the non-lipophilic, non-polymeric excipient is selected from the group consisting of lactose, maltose, dextrose, sucrose, fructose, non-polymeric carbohydrate, alpha-hydroxy acid, mannitol, sorbitol, xylitol, dicalcium phosphate, calcium sulfate, lactitol, glucose, sodium lauryl sulfate, derivatives of the foregoing, and combinations thereof.

17. The composition of claim 2, wherein the composition is substantially free of a release rate-retarding polymer.

18. The composition of claim 2, wherein the composition is substantially free of a release rate-retarding lipophilic excipient.

19. The composition of claim 2, wherein the composition is substantially free of pharmaceutical polymers, waxes, fats, fatty acids, and/or fatty alcohols.

20. The composition of claim 2, wherein the composition provide an extended release of macrolide antibiotic in the absence of a release rate-retarding polymer.

21. The composition of claim 2, wherein the composition has a hardness of about 5-40 Kp.

22. The composition of claim 2 further comprising a glidant present in an amount of about 0.01-5% by weight of the composition.

23. The composition of claim 2 further comprising a lubricant present in an amount of about 0.01-5% by weight of the composition.

24. The composition of claim 2 further comprising a wetting agent present in an amount of about 0.1-10% by weight of the composition.

25. A compressed extended release solid pharmaceutical composition comprising:
a) a macrolide antibiotic present in an amount of about 40-70% by wt. of the composition;
b) one or more surfactants present in an amount of up to about 10% by wt. of the composition;
c) one or more non-lipophilic, non-polymeric excipients present in an amount of about 20-60% by wt. of the composition; and
d) optionally one or more pharmaceutically acceptable excipients; wherein
the surfactant and the non-lipophilic, non-polymeric excipient cooperate to provide an extended release of macrolide antibiotic when the composition is exposed to an aqueous environment of use.

26. The composition of claim 25, wherein the pharmaceutical composition is made by mixing together and then compressing a granular composition and other functional excipients.

27. The composition of claim 26, wherein the granular composition comprises the macrolide antibiotic, one or more surfactants and optionally one or more non-lipophilic, non-polymeric excipients, and wherein the composition further comprises a binding 15 composition.

28. The composition of claim 27, wherein at least one non-lipophilic, non-polymeric excipient is disposed within the granular composition.

29. The composition of claim 27, wherein at least one non-lipophilic, non-polymeric excipient is disposed within the binding composition.

30. The composition of claim 27, wherein a non-lipophilic, non-polymeric excipient is disposed within the granular composition and the binding composition.

31. The composition of claim 27, wherein the surfactant is disposed within the granular composition and the binding composition.

32. The composition of claim 25, wherein the surfactant has an HLB value of at least 10.

33. The composition of claim 25, wherein the surfactant is selected from the group consisting of sodium lauryl sulfate, aerosil OT, cholic acid, salts of cholic acid, and combinations of the foregoing.

34. The composition of claim 25, wherein the macrolide antibiotic is selected from the group consisting of Amphotericin B, antimycin A, azithromycin, brefeldin A, candicidin, clarithromycin, dirithromycin, erythromycin, josamycin, kitasamycin, lucensomycin, maytansine, mepartricin, miocamycin, natamycin, nystatin, oleandomycin, roxithromycin, rutamycin, sirolimus spiramycin, tacrilomus, troleandomycin, tylosin, and derivatives thereof.

35. The composition of claim 25, wherein the macrolide antibiotic is present in an amount of about 100-1000 mg.

36. The composition of claim 25, wherein the non-lipophilic, non-polymeric excipient is a monosaccharide or disaccharide.

37. The composition of claim 25, wherein the non-lipophilic, non-polymeric excipient is selected from the group consisting of lactose, maltose, dextrose, sucrose, fructose, non-polymeric carbohydrate, alpha-hydroxy acid, mannitol, sorbitol, xylitol, dicalcium phosphate, calcium sulfate, lactitol, glucose, sodium lauryl sulfate, derivatives of the foregoing, and combinations thereof.

38. The composition of claim 25, wherein the composition is substantially free of a release rate-retarding polymer.

39. The composition of claim 25, wherein the composition is substantially free of a release rate-retarding lipophilic excipient.

40. The composition of claim 25, wherein the composition is substantially free of pharmaceutical polymers, waxes, fats, fatty acids, and/or fatty alcohols.

41. The composition of claim 25, wherein the composition provides an extended release of macrolide antibiotic in the absence of a release rate-retarding polymer.

42. The composition of claim 25, wherein the composition has a hardness of about 5-40 Kp.

43. The composition of claim 25 further comprising a glidant present in an amount of about 0.01-5% by weight of the composition.

44. The composition of claim 25 further comprising a lubricant present in an amount of about 0.01-5% by weight of the composition.

45. The composition of claim 25 further comprising a wetting agent present in an amount of about 0.1-10% by weight of the composition.

46. A method of treating a bacterial infection in a host, said method comprising administering to host the composition of claim 1.

47. A method of treating a bacterial infection in a host, said method comprising administering to the host the composition of claim 25.
